(19) United States
(12) Plant Patent Application Publication
(10) Pub. No.: US 2009/0271900 P1
(43) Pub. Date: Oct. 29, 2009

(54) Cynodon dactylon plant named 'LEG13A'

(50) ;

(76) Inventor: Todd Layt, Clarendon (AU)

Correspondence Address: Sam R. McCoy Jr., P.O. Box 2108, Mount Pleasant, SC, 29465, US

(21) Appl. No.: 12/386,193

(22) Filed: Apr. 16, 2009

(30) Foreign Application Priority Data
Apr. 28, 2008 (AU) 2008/110

(51) Int. Cl. A01H 5/00

(52) U.S. Cl. PLT/388

(57) ABSTRACT

A new and distinct cultivar of Cynodon dactylon plant named ‘LEG13A’, characterized by its fast speed of growth, very low seed head production, medium-strong plant density, strong sod strength, dark green leaf color and freedom from disease.

### LATIN NAME OF GENUS AND SPECIES OF PLANT CLAIMED

Cynodon dactylon

### VARIETY DENOMINATION

‘LEG13A’

### BACKGROUND OF THE INVENTION

The present invention relates to a new and distinct vigorous-growing perennial variety of Cynodon dactylon, which has been given the variety denomination of ‘LEG13A’. Its market class is that of a turf grass. ‘LEG13A’ is intended for use as a lawn grass in gardening, landscaping, sports fields and amenity horticulture.

The new Cynodon dactylon cultivar is a seedling selection discovered in a controlled planting of open pollinated Cynodon dactylon ‘C1’, (not patented) in a controlled environment at Clarendon, New South Wales, Australia. In 2005 approximately 1000 seedlings were germinated from ‘C1’ and grown to maturity. Seed were collected from these plants and in winter 2006 50 seedlings from this second generation were grown to maturity. These were each reproduced vegetatively by cuttings and grown as a replicated trial in order to assess traits of these 50 plant lines including speed of growth, sod strength, low seed head production, leaf colour and freedom from disease. Other proprietary breeding lines and commercial cultivars were included for comparison. In spring 2006 any line that produced abundant seed heads was eliminated from the program. From spring 2006 to autumn (fall) 2007 field trials were conducted of the remaining 8 low seed head producing lines from the breeding program. In March 2008 a single seedling was finally selected due to its fast speed of growth, strong sod strength, low seed head production, dark green leaf colour and freedom from disease.

The final selection designated ‘LEG13A’ was considered to be greatly improved compared to other varieties of Cynodon dactylon with great suitability for use as a lawn grass in gardening, landscaping, sports fields and amenity horticulture. The low seed head production reduces incidence of seed germiination in gardens, thus reducing weed potential and also means reduced mowing frequency in landscape and turf applications compared to higher seed head producing varieties. Less seed head production also means the plant put energy into vegetative, not reproductive growth and therefore recovers better from wear in sports field applications than higher seed head producing varieties. The reduced seed head production of ‘LEG13A’ also reduces the chance of invasive weed occurrence of this plant in native plant areas.

‘LEG13A’ was first asexually propagated by cutting and division of stolons in 2008 in Clarendon, New South Wales, Australia. ‘LEG13A’ has since been further asexually propagated by means of cutting and division of stolons. The distinctive characteristics of the variety have remained stable and true to type through successive cycles of asexual propagation.

### SUMMARY OF THE INVENTION

The following characteristics of the new cultivar have been repeatedly observed and can be used to distinguish ‘LEG13A’ as a new and distinct cultivar of Cynodon dactylon plant:
1. Fast speed of growth; and
2. very low seed head production; and
3. Medium-strong plant density; and
4. Strong sod strength; and
5. Dark green leaf color; and
6. Freedom from disease

Plants of the new cultivar differ from plants of the Cynodon dactylon parent ‘C1’ primarily in having a very low seed head production whereas ‘C1’ has a high rate of seed head production.

An application for plant breeders' rights for variety ‘LEG13A’ has been lodged with the Australian Plant Breeders' Rights Office, and was first gazetted on 28 Apr. 2008 under Application No. 2008/110.

### BRIEF DESCRIPTION OF THE PHOTOGRAPHS

The accompanying photographs show, as nearly true as it is reasonably possible to make the same in color illustrations of this type, typical foliage and growth characteristics of the new cultivar. Colors in the photographs differ slightly from the color values cited in the detailed description, which accurately describes the colors of ‘LEG13A’.

FIG. 1 illustrates a ‘LEG13A’ plant in a 200 mm pot grown for approximately 10 months in an outdoor environment showing plant growth 10 weeks after pruning (mowing) in autumn in Clarendon, NSW, Australia.

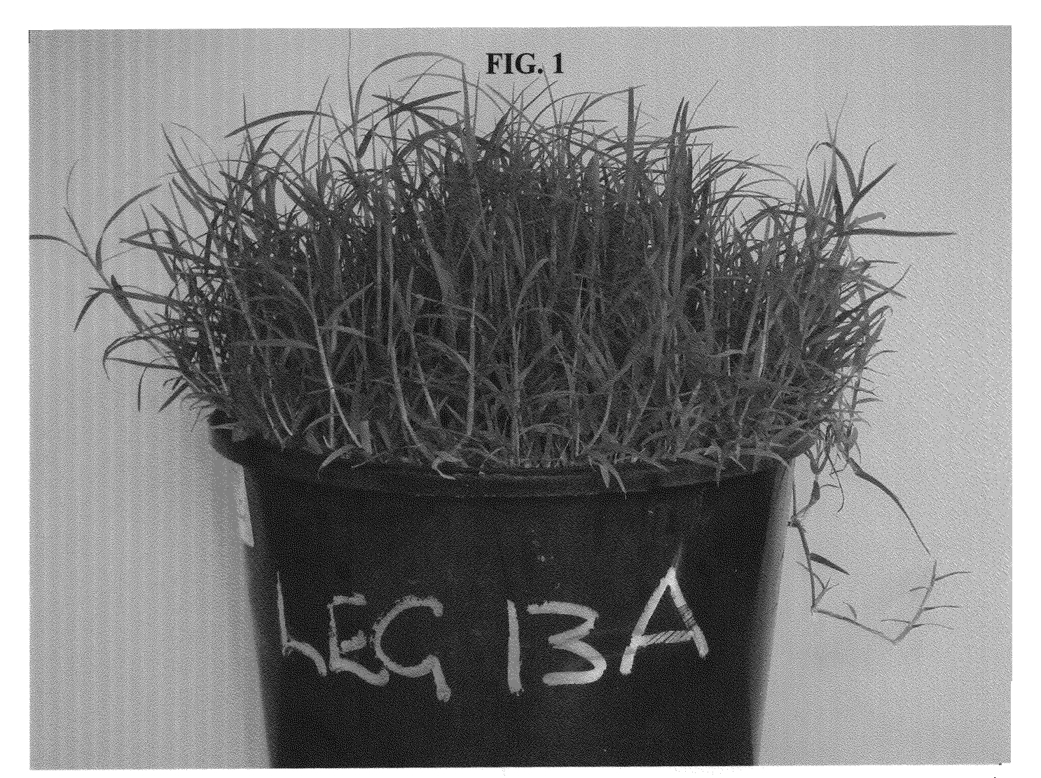

FIG. 2 illustrates the plant growth habit of a ‘LEG13A’ plant grown for approximately 10 months in an outdoor environment showing plant growth density, and leaf coloration 10 weeks after pruning (mowing).

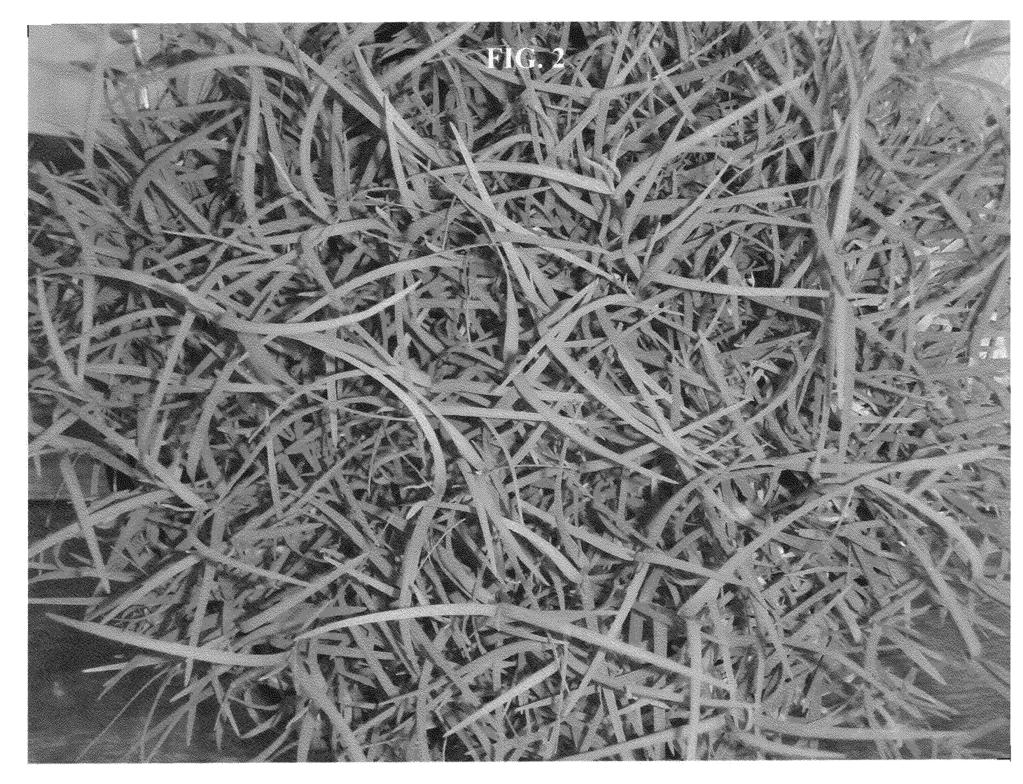

FIG. 3 illustrates ‘LEG13A’ stolon showing coloration and dimensions 10 weeks after pruning (mowing) in autumn in Clarendon, NSW, Australia.

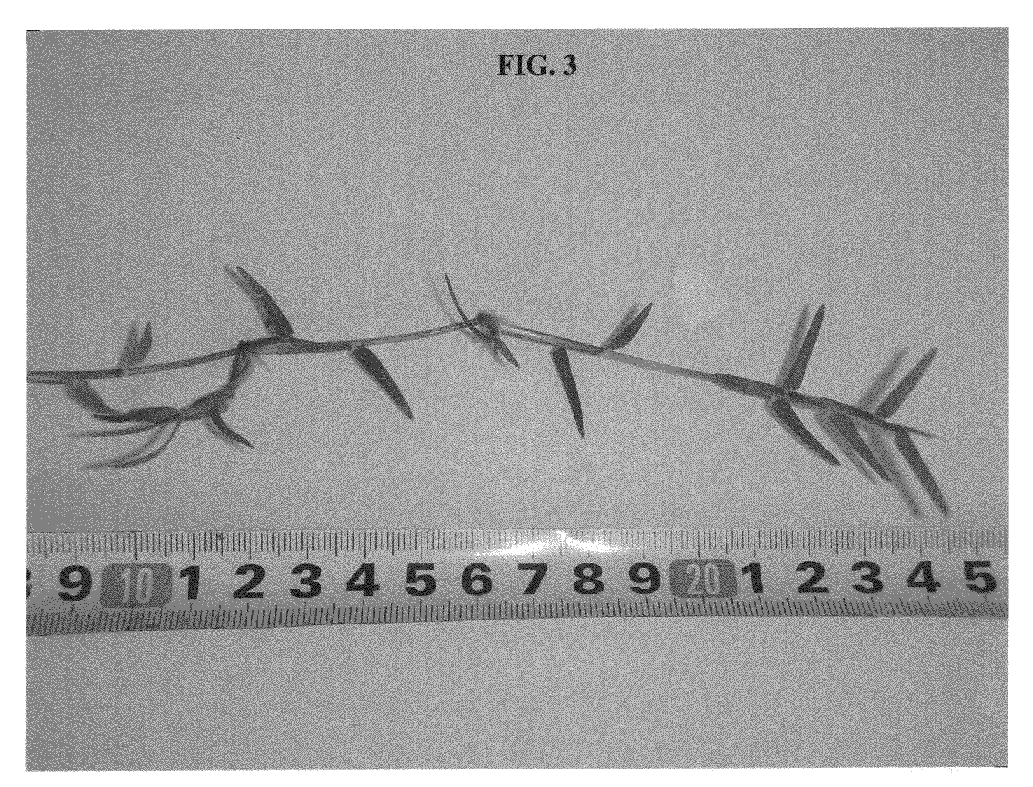

FIG. 4 illustrates ‘LEG13A’ stolon and. inflorescence relative dimensions in autumn in Clarendon, NSW, Australia

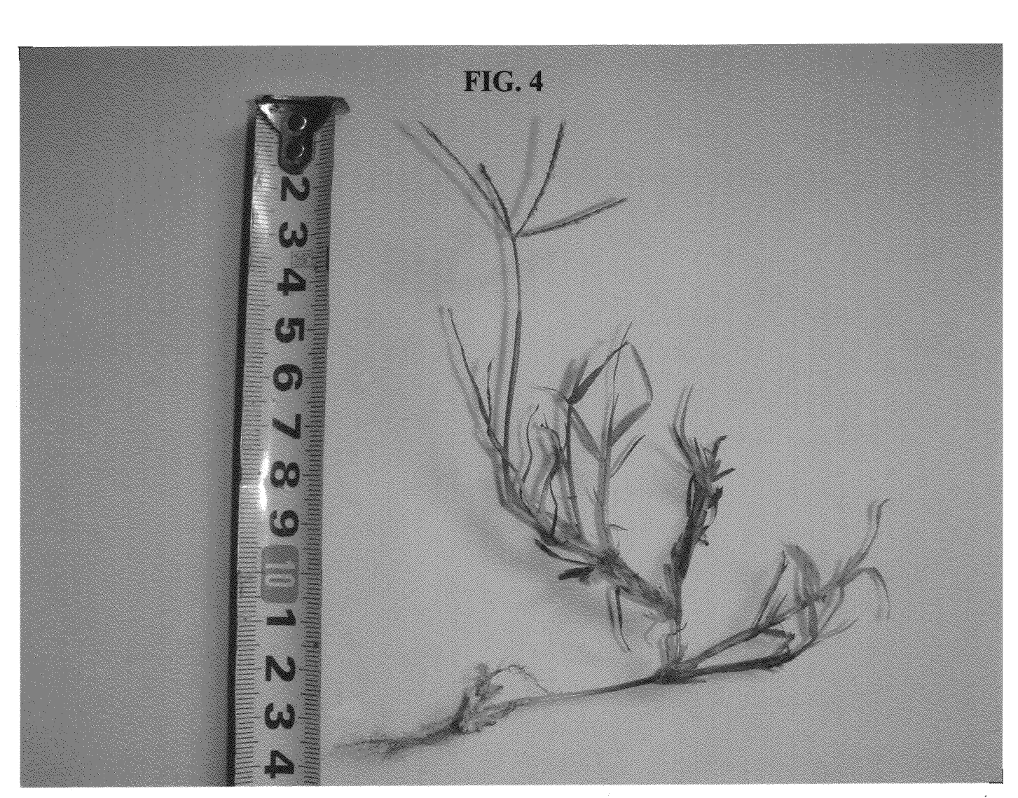

FIG. 5 illustrates ‘LEG13A’. inflorescence detail in autumn in Clarendon, NSW, Australia

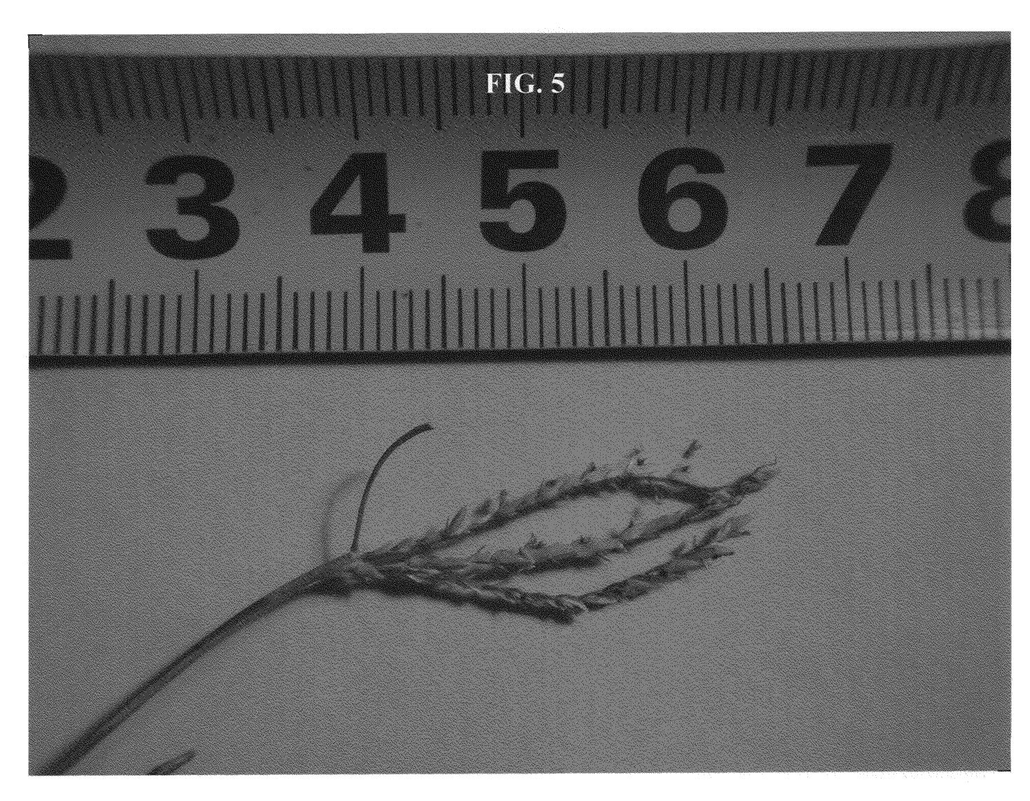

### DETAILED BOTANICAL DESCRIPTION

The following is a detailed botanical description of a new and distinct variety of an Cynodon dactylon ornamental plant known as ‘LEG13A’. Plant observations were made on plants grown in Clarendon, New South Wales, Australia. Unless indicated otherwise, the descriptions disclosed herein are based upon observations made from May 2008 of mature ‘LEG13A’ plants grown in nursery pots in greenhouse and outdoor growing areas with day temperature ranging from 25° C. to 35° C., night temperatures ranging from 10° C. to 15° C., and light levels ranging from 6 to 8 klux. Plants were grown for about 24 weeks with one plant per 20 cm container. Plants were trimmed 10 weeks before assessment. Those skilled in the art will appreciate that certain characteristics will vary with older or, conversely, younger plants. ‘LEG13A’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light quality, light intensity, day length, cultural conditions and the like. Color notations are based on The Royal Horticultural Society Colour Chart, of The Royal Horticultural Society, London, 2007 edition.
Botanical classification: Cynodon dactylon cultivar ‘LEG13A’.
Parentage: Parents — Cynodon dactylon breeder code ‘C1’
Propagation: Type — Asexual propagation of cuttings from stolons and divisions of plants.
Technical description of the variety: The description of the variety below is taken from a pot trial conducted in the Summer 2008 to autumn 2008 in Clarendon, New South Wales, Australia. Plants were six-months old at the time of observation. Further observations were made in March 2009 to confirm colors with plants with 10 weeks regrowth post mowing.
Plant characteristics: vigorous-growing perennial, stoloniferous grass, culms decumbent, habit prostrate becoming erect when flowering.
Stolon: Roots at nodes, length long (mean 30 cm) internode length (4th from tip) medium (mean 24 mm), diameter of internode medium (mean 1.5 mm), color of internode when exposed to sunlight 177B, diameter of node medium (mean 2.0 mm), branching medium, subtended by a leafy sheath with length 6 to 9 mm.
Leaf: attachment cauline, attitude semi-erect, length short (mean 13.7 mm), width broad (mean 2.8 mm), color of blade green (RHS 146A), leaf veins parallel and obscure, shape linear-triangular, apex narrow acute, leaf margin entire.
Ligule: Ligule is a fringe of hairs, not prominent.
Awns: Absent.
Inflorescence: Digitate raceme with 3 to 4 spikes of length 25-30 mm and color green (RHS 146A); length of peduncle short (mean 60 mm), color of peduncle green (RHS 146A).
Glumes: color grayed yellow (RHS 160C-D) and fading to grayed white (approximately RHS 155D), length 1.5-2.0 mm
Lemmas: color grayed yellow (RHS 160C-D) and fading to grayed white (approximately RHS 155D), length 1.5 mm.
Anther color: grayed yellow approximately (RHS 160C-D) changing to grayed orange (RHS 174A), length about 1.2 mm.
Stigma color: white 155D.
Comparison of ‘LEG13A’ with other varieties of Cynodon dactylon: Plants of the new cultivar differ from plants of the Cynodon dactylon parent ‘C1’ primarily in having a very low seed head production whereas ‘C1’ has a high rate of seed head production. A sample taken in autumn 2008 at Clarendon, New South Wales, Australia showed an average of 0.6 inflorescences (seed heads) per plant for ‘LEG13A’ whereas ‘C1’ was found to have an average of 13 inflorescences per plant. ‘LEG13A’ also differs from ‘C1’ due to a long stolon length and a medium plant density whereas ‘C1’ has a short to medium stolon length with a strong plant density. The very low seed head production of ‘LEG13A’ differs to the varieties ‘Greenlees Park’, ‘RR1’, ‘Oz-E-Green’, ‘Riley's Super Sport’, ‘TL1’, ‘JT1’ and ‘LTA’ which have medium to high production of seed heads. ‘LEG13A’ also differs to the varieties ‘Winter Gem’, ‘Wintergreen’ and ‘Riley's Evergreen’ which have low to medium seed head production. ‘LEG13A’ has a medium stolon internode length which differs from the varieties ‘Santa Ana’, ‘Plateau’ and ‘Grand Prix’ which all have very short stolon internode lengths. ‘LEG13A’ has a medium plant density which differs from the varieties ‘CT2’ and ‘Windsor Green’ which have strong plant densities.
Cultural notes: ‘LEG13A’ has been observed to have good turfgrass performance to USDA zone 7b. ‘LEG13A’ has been tested for heat tolerance to 47° Celsius. ‘LEG13A’ grows well in very sandy and heavy silt soil conditions. ‘LEG13A’ has shade tolerance typical of Cynodon dactylon. ‘LEG13A’ competes well with invasive weeds. It strikes well when being stolonised, and transplants well as sod.
Disease resistance: ‘LEG13A’ has no known disease susceptibilities and is considered typical of the species for pest and disease tolerance.

What is claimed is:

1. A new and distinct cultivar of Cynodon dactylon plant named ‘LEG13A’, substantially as herein shown and described.
